(19) United States
(12) Patent Application Publication
(10) Pub. No.: US 2005/0106689 A1
(43) Pub. Date: May 19, 2005

(54) Synthesis of aminocrotonates

(75) Inventors: Max Braun, Wedemark (DE); Saskia Braukmueller, Sarstedt (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Fluor und Derivate GmbH, Hannover (DE)

(21) Appl. No.: 10/944,464

(22) Filed: Sep. 20, 2004

Related U.S. Application Data
(63) Continuation of application No. PCT/EP03/02228, filed on Mar. 5, 2003.

(30) Foreign Application Priority Data
Mar. 21, 2002 (DE) 102 12 525.2
Dec. 20, 2002 (DE) 102 59 911.4

(51) Int. Cl. C12P 13/04; C07C 227/08

(52) U.S. Cl. 435/106; 562/443; 562/556

(57) ABSTRACT

3-aminocrotonate compounds, e.g. diesters, which are substituted on the C-4 atom by at least two chlorine and/or fluorine atoms, are produced by reacting corresponding acetoacetate compounds with ammonia and primary and secondary amines while simultaneously or subsequently eliminating water. The reaction is carried out in the presence of onium salts formed from primary or secondary amines and carboxylic acids. Preferably, two phases are formed; one phase containing the crotonate compound product with a good degree of purity and the other phase containing the onium salt and water.

### CROSS REFERENCE TO RELATED APPLICATIONS

This application is a continuation of international application No. PCT/EP03/02228, filed Mar. 5, 2003, designating the United States of America and published in German as WO 03/080562 on Oct. 2, 2003, the entire disclosure of which is incorporated herein by reference. Priority is claimed based on Federal Republic of Germany patent application Nos. DE 102 12 525.2, filed Mar. 21, 2002 and DE 102 59 911.4, filed Dec. 20, 2002.

### BACKGROUND OF THE INVENTION

The invention relates to a simplified method of synthesizing 3-aminocrotonate compounds, which are substituted at the C-4 atom by at least two chlorine atoms and/or fluorine atoms.

Such halogen-substituted 3-aminocrotonates can be used as intermediates, for example, for dyes or photographic materials, such as those listed in the introduction of published Japanese patent application No. JP 05-140060. Aminocrotonates may also be used as intermediates for agricultural chemicals or active ingredients of pharmaceutical products.

The synthesis of these compounds is known. For example, European patent application No. EP 808,826 discloses their synthesis from haloacetoacetates and an ammonium salt. When these materials are heated, the desired product is formed. It is worked up by distillation or by solvent/solvent or solvent/water extraction.

Published PCT application No. WO 99/24350 discloses the synthesis of halogenated aminocrotonates from haloacetoacetates and amines. The water, formed during the reaction, is removed by an entraining agent or by passing an inert gas through the reaction mixture.

### SUMMARY OF THE INVENTION

It is an object of the present invention to provide an improved method for synthesizing halogenated aminocrotonates.

This and other objects are achieved in accordance with the present invention by providing a method of synthesizing a 3-aminocrotonate compound which is substituted on the C-4 atom by at least two halogen atoms selected from the group consisting of chlorine and fluorine, said method comprising reacting an acetoacetate compound, which is substituted on the C-4 atom by at least two halogen atoms selected from the group consisting of chlorine and fluorine, with ammonia or a primary or secondary amine in the presence of an “onium” salt formed from a primary, secondary, tertiary or quaternary amine and a carboxylic acid, and simultaneously or subsequently eliminating water.

The method of the invention for synthesizing 3-aminocrotonates, substituted at the C-4 atom by at least 2 chlorine atoms and/or fluorine atoms, by reacting acetoacetate compounds, substituted at the C-4 atom by at least 2 chlorine atoms and/or fluorine atoms, with ammonia, primary or secondary amines, water being split off simultaneously or subsequently, provides for the synthesis in the presence of “onium” salts, which are formed from primary, secondary, tertiary or quaternary amines and carboxylic acids. In this connection, it is clear to those, skilled in the art that, in relation to “quaternary amine”, a more correct expression would be “quaternary ammonium cations and anions of carboxylic acids”. A (free) amine, for reacting with the ester for the purpose of forming the crotonate, as well as the “ionium” salt is used for the method of the invention.

The method can be carried out in two embodiments, namely, so that one phase is formed or that two phases are formed. The invention will be described initially with reference to the embodiment in which two phases are formed, one phase containing the 3-aminocrotonate product compound. The water of reaction is in the phase containing the “onium” salt and cannot react further with the product in an undesirable manner (saponification). This embodiment is therefore particularly advantageous for this reason, as well as because a two-phase mixture can be worked up easily.

Preferably “onium” salts of ammonia and, in particular, salts of primary or secondary amines are used. The use of those “onium” salts, the cation of which corresponds to the amine used for the synthesis of the aminohalogencrotonate, is particularly advantageous.

The method is highly suitable for compounds in which the C-4 atom is substituted by two fluorine atoms, three fluorine atoms or a chlorine atom and two fluorine atoms.

Preferably, the method of the invention is used for the synthesis of aminohalaogencrotonate compounds, such as those disclosed in the aforementioned European patent application. The method is one for the synthesis of a 3-amino-4,4,4-trihalogencrotonate of formula (I)
X₃C—C(NR¹R²)═C(R)—C(A)B (I)
in which
X represents fluorine or chlorine,
A represents O, S or NR⁵,
B represents R⁶, OR⁶, SR⁶ or NR³R⁴
R, R¹, R², R³, R⁴, R⁵ and R⁶, independently of one another, represent H, C1 to C6 alkyl, C2 to C6 alkenyl, C2 to C6 alkinyl, phenyl or phenyl(C1 to C6)alkyl or C1 to C6 alkyl, C2 to C6 alkenyl, C2 to C6 alkinyl, phenyl or phenyl(C1 to C6)alkyl substituted with one or more groups selected independently from halogen, CN, NO₂, C1 to C6 alkyl, C2 to C6 alkenyl, C2 to C6 alkinyl, phenyl, phenyl(C1 to C6)alkyl, C1 to C6 alkoxy, C2 to C6 alkenyloxy and phenoxy, or R¹ and R², and R³ and R⁴, may be connected in each case independently of one another with the nitrogen, to which they are connected, so that a five-membered, six-membered or seven-membered heterocyclic ring is formed, or when A represents NR⁵ and B represents OR⁶ or SR⁶, R⁵ and R⁶ may be connected with the A=C−B group, to which they are linked, so that they form a five-membered, six-membered or seven membered heterocyclic ring, or when A represents NR⁵ and B represents NR³R⁴, R³ or R⁴ and R⁵ may be connected with the A=C−B group, to which they are linked, so that a five-membered, six-membered or seven-membered heterocyclic ring is formed, from ammonia or the corresponding amines and the corresponding β-ketocarboxylic acid. Preferably, CX₃ is CF₃, CF₂Cl or CHF₂. R¹ and R² are the same or different and preferably represent H or C1 to C4 alkyl. A preferably is O or S and especially O. R preferably is H or C1 to C4 alkyl. B preferably is OR⁶ or SR⁶. R³, R⁴ and R⁵ preferably are H or C1 to C3 alkyl. R⁶ preferably is C1 to C3 alkyl and may optionally be substituted by one more fluorine atoms.

In the following, “onium” cations, which are used as “onium” salts, are explained in greater detail.

The concept, “onium”, preferably represents cations with a positively charged nitrogen atom, for example, protonated aromatic nitrogen bases such as. pyridinium or protonated alkylammonium, dialkylammonium or trialkylammonium cations or ammonium compounds substituted by cycloalkyl or cycloaliphatic nitrogen bases, such as pyridinium or quaternary ammonium cations. These are protonated cations or quaternary cations of nitrogen.

“Onium” cations of nitrogen, having the formula R′R″R′″R″″N⁺ are very suitable. R′, R″, R′″ and R″″ independently of one another represent hydrogen, alkyl with 1 to 20 carbon atoms, aryl or aralkyl. R′ and R″ or R′″ and R″″, or R′, R″ and R′″ or R′, R″, R″″ and R″″ may also, optionally, with inclusion of the nitrogen atom, form saturated or unsaturated ring systems. “Aryl” refers here, in particular, to phenyl or phenyl, substituted by one or more C1 to C2 alkyl groups. Outstandingly suitable are salts, in which “onium” represents ammonium, pyridinium or R^{1′}R^{2′}R^{3′}R^{4′}N⁺, in which R^{1′}. R^{2′}, R^{3′} and R^{4′} independently of one another represent hydrogen, alkyl with 1 to 15 carbon atoms, phenyl or benzyl. Examples of such cations include pyridinium, piperidinium, N-methylpiperidinium, anilinium, benzyltriethylammonium and triethylammonium.

Protonated cations of amines, substituted by hydroxy groups, especially of cycloaliphatic amines, particularly hydroxy-substituted piperidines and N-(C1 to C4) alkylpiperidines, may also be used. Piperidines, substituted at the C4 atom, such as 4-hydroxypiperidine, N-methyl-4-hydroxpiperidine, N-ethyl-4-hydroxypiperidine and N-propyl-4-hydroxy-piperidine are suitable.

Also readily usable are protonated cations of pyridine, which are substituted by 1, 2, 3 or more alkyl groups with one to four carbon atoms. Protonated cations of pyridine, which are substituted by 1, 2 or 3 methyl or ethyl groups, are preferred here. Cations of picoline, lutidine and collidine, especially of 2-picoline, are preferred.

Cations of amines which are disclosed in published U.S. patent application No. U.S. 2004/097758 A1 (=DE 101 04 663), the disclosure of which is incorporated herein by reference, can also be used. These are “onium” cations based on monocyclic or bicyclic compounds with at least 2 nitrogen atoms, at least 1 nitrogen atom being incorporated in the ring system.

For example, “onium” cations, based on monocyclic compounds may be used. These are saturated or unsaturated five-membered, six-membered or seven-membered ring compounds. At least one nitrogen atom is incorporated in the ring. A further nitrogen atom may also be incorporated in the ring system. Alternatively or additionally, the ring may also be substituted by one or more amino groups. Dialkylamino groups, in which the alkyl groups may be the same or different and contain one to four carbon atoms, are preferred. The amino group may also represent a saturated ring system, such as a piperidino group. Representatives monocylic ring systems, which can be used readily, include dialkylaminopyridine, dialkylaminopiperidine and dialkylaminopiperazine.

“Onium” cations of bicyclic compounds may also be used. Here also, one, two or more nitrogen atoms may be integrated in the ring system. The compounds may be substituted by one or more amino groups. Preferred once again are dialkylamino groups, the alkyl groups being the same or different and having one to four carbon atoms or, together with the nitrogen atom, forming a saturated ring system, such as the piperidinyl group.

It is clear from the foregoing that, in the case of this embodiment, at least two nitrogen atoms in the compounds, which can be used, must have basic properties and, depending on the nature of the bonds, be linked to two or three carbon atoms.

Especially preferred are “onium” cations of bicyclic amines, especially of 1,5-diaza-bicyclo[4.3.0]-5-nonene (DBN) and 1,8-diazabicyclo[5.4.0]-7-undecene (DBU).

The anions of the “onium” salts are anions of carboxylic acids. Preferred carboxylic acids, the anions of which are used, are those with one to six carbon atoms. Particularly advantageous are aliphatic, branched or linear carboxylic acid anions, especially those with 1 to 4 carbon atoms, especially if they are substituted by at least 1 halogen atom. Outstandingly suitable our anions, which are derived from acetic acid or propionic acid, as well as anions, which are derived from acetic acid or propionic acid and are substituted by at least 1 fluorine atom. Anions of acetic acid, propionic acid, monofluoroacetic acid, difluoroacetic acid, trifluoroacetic acid, chlorodifluoroacetic acid and perfluoropropionic acids are particularly suitable.

The “onium” salts may be synthesized from an amine and a carboxylic acid, optionally with an excess of the latter. It is also possible to synthesize the “onium” salts in the reactor in situ. For example, recycled “onium” salt phase may be saturated with amine and the carboxylic acid may be added directly to the reactor in the desired amount.

An excess of carboxylic acid may be used for the 1-phase as well as the 2-phase variation of the inventive method. Accordingly, the molar ratio of amine to carboxylic acid in “onium” salt may range from 1:1 up to 1:8 or even more.

The reaction is carried out at a temperature ranging from 40° to 140° C., depending on the rate at which water is split off.

The molar ratio of “onium” salt to acetoacetate ester advantageously falls within the range 1:0.5 to 1:40 and especially within the range of 1:0.5 to 1:25 and particularly within the range of 1:1 to 1:25.

The range, in which two phases are formed, varies depending on the “onium” salt used and on the crotonate synthesized. For example, the amine is used only in an amount, which is sufficient for up to 90 mole percent of the acetoacetate, or for some amines, up to 100 or even 140 mole percent. However, the formation of two phases can be followed very well visually and those, skilled in the art, can easily estimate whether the formation of two phases is optimum for the particular reaction. For example, the “onium” salt may be transferred to the reactor, after which the acetoacetate compound, followed gradually by the amine, are added. The amounts of amine, for which there is one phase, and the amounts of amine, for which there are two phases, can be recognized easily.

Water is formed during the reaction of ammonia or amine with acetoacetate. The water accumulates in the salt phase and can be removed by vacuum distillation, by passage of an inert gas such as nitrogen (or air, if desired), by membrane separation or by other methods of removing water. For example, inorganic oxide absorbents (drying agents) can be used. Silica-based absorbents, such as Sicolith 400 and AF 125 (AF stands for “aluminum-free”), are very suitable and may be obtained as drying pearls Engelhard Process Chemicals GmbH, Nienburg, Germany.

A noticeable difference in density is advantageous for the formation of two phases. The density of the crotonate formed is higher than the density of the “onium” salt, so that an “onium” salt with a lower density is advantageous. For this reason, salts with carboxylic acids, which are not halogenated, can readily be used.

The crotonate can be removed from the salt phase by decantation or by some other means of phase separation, such as allowing the lower phase to run off.

The method of the invention yields a very pure crotonate even without additional distillation. The salt phase can be reused, the water of reaction being removed occasionally. The method is very selective.

The other embodiment is carried out in one phase. Here also, someone, skilled in the art, can easily determine the one-phase range for a particular reaction. As is already described above, the “onium” salt can be added to the reactor first, followed by the acetoacetate and then gradually by the amine. The range, in which the reaction mixture forms a single phase, can then be recognized.

The one-phase, as well as the two-phase embodiment can be carried out batchwise or continuously; the two-phase embodiment can be carried out particularly well.

The following examples are intended to describe the invention further without limiting its scope.

### EXAMPLES

Explanation of the abbreviations:

N-Me-EATC: Ethyl 3-methyl amino-4,4,4-trifluorocrotonate
DBN: 1,5-Diaza-bicyclo[4.3.0]-5-nonene
TFA: Trifluoroacetic acid
AcOH: Acetic acid
ETFAA: Ethyl 4,4,4-trifluoroacetate
N-Me-N-Me-EATC N-Methyl-3-methylamino-4,4,4-triluorocrotonamide

### Example 1

### Synthesis of N-Me-EATC with DBN*2TFA Reaction: CF₃COCH₂CO₂C₂H₅ (=ETFAA)+CH₃NH₂→CF₃CNHCH₃CHCO₂C₂H₅

Formulation:

1. Preparation of DBN*2TFA

| | |
|---|---|
| 0.1 moles of DBN | 12.4 g |
| 0.2 moles of TFA | 22.8 g |

2. Preparation of N—Me-EATC:

| | |
|---|---|
| 1.5 moles of ETFAA | 276.17 g |
| 1.5 moles of CH₃NH₂ | 46.59 g |

Procedure:

The amine was added to a 500 ml flask and the TFA was added carefully dropwise at room temperature (exothermic reaction!). The temperature was then adjusted to approximately 50° C. and the ETFAA was added dropwise. The methylamine was then passed into the liquid at 85° C. (but only up to a conversion of approximately 80% of the ETFAA). When 75 mole percent of the methylamine had been added, the N-Me-EATC segregated as the main constituent of a second phase. Selectivity: 97.9% N-Me-EATC/2.1% N-Me-N-Me ATCA. The N-Me-EATC was then finally purified at a temperature of 62° to 64° C. and a pressure of 6 mbar (purity: 99.9% by GC area).

### Example 2

### Synthesis of N-Me-EATC with DBN*3TFA Reaction: CF₃COCH₂CO₂C₂H₅+CH₂NH₂→CF₃CNHCH₃CHCO₂C₂H₅

1. Preparation of DBN*3TFA

| | |
|---|---|
| 0.1 moles of DBN | 12.4 g |
| 0.3 moles of TFA | 34.2 g |

2. Preparation of N—Me-EATC:

| | |
|---|---|
| 1.5 moles of ETFAA | 276.17 g |
| 1.5 moles of CH₃NH₂ | 46.59 g |

### Example 2.1

The amine was added to a 500 ml flask and the TFA was carefully added dropwise at room temperature (exothermic reaction!). The temperature was then adjusted to approximately 50° C. and the ETFAA was added dropwise. The methylamine was then passed into the liquid at 85° C. (but only up to an approximately 80%/80 mole percent of the methylamine). When 75 mole percent of the methylamine had been added, two phases were formed. GC analysis of the organic upper phase revealed a selectivity of 97.8% to N-Me-EATC and 2.2% to N-Me-N-Me EATC. According to a Karl Fischer analysis, the catalyst phase meanwhile contained 7.4% by weight=1.05 moles of water.

### Example 2.2

### Repetition of the Experiment with the Catalyst Phase Obtained from the Reaction Above

The catalyst phase remained in the flask and the experiment was continued. Once again, 1.5 moles of ETFAA were added and 80 mole percent of 1.5 moles of CH₃NH₂ were passed in once again at 85° C. At 65 mole percent, two phases were formed. GC analysis of the upper, organic phase revealed a selectivity of 98.7% to N-Me-EATC and 1.3% to N-Me-N-Me-EATC. According to a Karl Fischer analysis, the catalyst phase contained 14.4%=1.05 moles of water. Excellent selectivity to NMe-ETAC was observed in spite of this high water content.

### Example 2.3

### Repetition of the Experiment with the Catalyst Phase Obtained from the Reaction Above

The method of 2.2 was repeated. At 65 mole percent, two phases were formed. GC analysis of the upper, organic phase revealed a selectivity of 98.9% to N-Me-EATC and 0.9% to N-Me-N-Me-EATC. According to a Karl Fischer analysis, the catalyst phase contained 18.4%=1.05 moles of water. Excellent selectivity to NMe-ETAC was observed in spite of this high water content.

### Example 3

### Removal of Water from DBN*3TFA+H₂O

Formulation:

| | |
|---|---|
| 12.42 g | (0.1 moles) of DBN |
| 34.20 g | (0.3 moles) of TFA |

+20% by weight of water

Procedure:

The amine was transferred to a 250 ml 3-neck flask and the TFA was carefully added dropwise, after which the water was added. The temperature was then raised to about 80° C. and nitrogen was blown in through a 1/8 inch tube at the rate of about 15 to 20 l/h). Excess water was taken off at the stillhead. Nitrogen was blown in for 5 hours and a sample was taken from the flask every hour. In addition, the distillate was weighed after every hour.

Sampling:

| | | | | |
|---|---|---|---|---|
| SB009501 | After 1 hr | 13.5% H₂O | 57.4% TFA | 0.0 g distillate |
| SB009502 | After 2 hr | 7.3% H₂O | 65.6% TFA | 0.0 g distillate |
| SB009503 | After 3 hr | 0.12% H₂O | 61.8% TFA | 0.9 g distillate |
| SB009504 | After 4 hr | 0.09% H₂O | 72.7% TFA | 2.1 g distillate |
| SB009505 | After 5 hr | 0.02% H₂O | 71.3% TFA | 3.0 g distillate |

The example shows that water can be removed from the salt phase by an inert gas (which could also be air).

### Example 4

### Removal of Water of Reaction from the Onium Phase (Catalyst Phase) by Distillation

The reaction mixture had the following initial composition:
12.4 g (0.1 moles) of DBN
34.2 g (0.3 moles) of TFA
9.32 g (20% by weight) of water.

A vacuum of 20 mbar was applied and the temperature was increased to 60° C. After 1 hour, the amount of water had been decreased from the original 20% by weight to <1% by weight; the ratio of DBN to TFA was still 1:3, that is, the TFA had remained completely in the catalyst mixture. However, the water had been distilled of selectively.

The example shows that water can be removed from the salt phase also by distillation without changing the ratio of DBN to TFA.

### Example 5

### Batchwise Synthesis of N-Me-EATC with DBN*6AcOH (5 Mole Percent of Catalyst)

Formulation:

6.21 g (0.05 moles) of DBN
18.02 g (0.3 moles) of AcOH (acetic acid)
184.11 g (1.0 mole) of ETFAA
31.06 g (1.0 mole) of CH₃NH₂
   Procedure:

The amine was transferred to a 500 ml multi-neck flask and the acetic acid was added dropwise (exothermic reaction up to 40° C.). The formulation turned solid during the dropwise addition. Later on, the “onium” salt formed was dissolved at about 45° C. The formulation was now maintained at about 50° C. and the ETFAA was added dropwise. The temperature was then raised to 85° C. and the methylamine was passed in. In order to remove excess water, nitrogen was bubbled into the formulation during the dropwise addition of the ETFAA and while the methylamine was being passed in. A two-phase range extended from 50 to 100 mole percent of methylamine. After the methylamine had been added, thermolysis was continued for 1.5 hours at 85° C. while nitrogen was passed through the formulation, after which the latter was distilled under vacuum. The selectivity was quantitative. At a temperature of 76° to 77° C. and a vacuum of 60 mbar, NME-EATC was isolated with a purity of 99%.

### Examples 6 to 11

### Synthesis of Ethyl N-methyl-4,4,4-trifluoroaminocrotonate

Example 5 was repeated with different “onium” salts. The range for the two-phase reaction and the yields are given in the following Table:

| | | | Selectivity |
|---|---|---|---|
| | | Phase Boundaries | N—ME-EATC |
| Experiment No. | Catalyst | (mole percent) | (%) |
| Example 6 | DBN*3FTA | 34-100 | 98 |
| | (5 mole %) | | |
| Example 7 | 2-picoline*3TFA | 34-100 | 98 |
| | (5 mole %) | | |
| Example 8 | DBN*3TFA | 20-102 | 98 |
| | (10 mole %) | | |
| Example 9 | DBN*3AcOH | 60-100 | 98 |
| | (10 Mole %) | | |
| Example 10 | 2-picoline*3TFA | 25-122 | 98 |
| | (5 mole %) | | |
| Example 11 | DBN*6TFA | 20-116 | 99 |
| | (5 mole %) | | |

It can be seen that “onium” salts can be used with different cations and anions and that the ratio of cation to carboxylic anion or carboxylic acid can be variable. Each of the examples 5 to 11 was repeated several times with always the same result. The example could also be carried out continuously, especially in the given two-phase range.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of synthesizing a 3-aminocrotonate compound which is substituted on the C-4 atom by at least two halogen atoms selected from the group consisting of chlorine and fluorine, said method comprising reacting an acetoacetate compound, which is substituted on the C-4 atom by at least two halogen atoms selected from the group consisting of chlorine and fluorine, with ammonia or a primary or secondary amine in the presence of an “onium” salt formed from a primary, secondary, tertiary or quaternary amine and a carboxylic acid, and simultaneously or subsequently eliminating water.

2. A method according to claim 1, wherein the concentration of the “onium” salt is selected so that two phases are formed, one phase containing the 3-aminocrotonate compound product.

3. A method according to claim 1, wherein the acetoacetate compound is a β-ketocarboxylic acid, and the synthesized 3-aminocrotonate compound corresponds to the formula (I):
X₃C—C(NR¹R²)═C(R)—C(A)B (I)
wherein
X represents fluorine or chlorine,
A represents O, S or NR⁵,
B represents R⁶, OR⁶, SR⁶ or NR³R⁴
R, R¹, R², R³, R⁴, R¹ and R⁶, independently of one another, represent H, C1 to C6 alkyl, C2 to C6 alkenyl, C2 to C6 alkinyl, phenyl, phenyl(C1 to C6)alkyl or C1 to C6 alkyl, C2 to C6 alkenyl, C2 to C6 alkinyl, phenyl, phenyl(C1 to C6)alkyl substituted with one or more groups selected independently from halogen, CN, NO₂, C1 to C6 alkyl, C2 to C6 alkenyl, C2 to C6 alkinyl, phenyl, phenyl(C1 to C6)alkyl, C1 to C6 alkoxy, C2 to C6 alkenyloxy and phenoxy, or
R¹ and R², and R³ and R⁴, may be connected in each case independently of one another with the nitrogen, to which they are connected, so that a five-membered, six-membered or seven-membered heterocyclic ring is formed, or
when A represents NR⁵ and B represents OR⁶ or SR⁶, R⁵ and R⁶ may be connected with the A=C−B group, to which they are linked, so that they form a five-membered, six-membered or seven membered heterocyclic ring, or
when A represents NR⁵ and B represents NR³R⁴, R³ or R⁴ and R⁵ may be connected with the A=C−B group, to which they are linked, so that a five-membered, six-membered or seven-membered heterocyclic ring is formed.

4. A method according to claim 3, wherein the synthesized 3-aminocrotonate is a 3-amino-4,4,4-trifluorocrotonate compound.

5. A method according to claim 4, wherein the synthesized 3-aminocrotonate compound is a 3-amino-4,4,4-trifluorocrotonate ester.

6. A method according to claim 1, wherein the “onium” salt corresponds to formula (II):
X—Y (II)
wherein
X represents a primary, secondary, tertiary or quaternary ammonium cation, and
Y represents a deprotonated anion of a C1 to C5 carboxylic acid or of a C1 to C5 carboxylic acid which is substituted by at least one fluorine atom.

7. A method according to claim 6, wherein Y represents the anion of a carboxylic acid selected from the group consisting of acetic acid, propionic acid, difluoroacetic acid, chlorodifluoroacetic acid, monofluoroacetic acid, trifluoroacetic acid and perfluoropropionic acid.

8. A method according to claim 6, wherein X represents a cation selected from the group consisting of DBN, DBU, 2-picoline, 4-picoline, 2,6-dimethylpicoline, pyridine, substituted pyridine, aniline and quinoline.

9. A method according to claim 1, wherein the synthesis of the 3-aminocrotonate compound is carried out at a temperature ranging from 60° to 140° C.

10. A method according to claim 1, further comprising removing water from an “onium” salt-containing phase by distillation or by using an inert gas or by treatment with an inorganic oxide drying agent.

11. A method according to claim 1, wherein up to 1.40 moles of the primary or secondary amine is used per mole of the acetoacetate compound.

12. A method according to claim 11, wherein up to 0.9 mole of the primary or secondary amine is used per mole of the acetoacetate compound.
